(19) United States
(12) Patent Application Publication
(10) Pub. No.: US 2026/0151265 A1
(43) Pub. Date: Jun. 4, 2026

(54) DEVICES FOR CANNULA INSERTION

(71) Applicant: Alcon Inc., Fribourg (CH)

(72) Inventors: Paul R. Hallen, Colleyville, TX (US); Grace Chuang Liao, Irvine, CA (US)

(21) Appl. No.: 19/394,027

(22) Filed: Nov. 19, 2025

Related U.S. Application Data
(60) Provisional application No. 63/726,848, filed on Dec. 2, 2024.

(51) Int. Cl. A61F 9/007

(52) U.S. Cl. CPC A61F 9/00754

(57) ABSTRACT

Embodiments of the present disclosure relate to cannula insertion systems for inserting a plurality of cannulas into a target tissue.

### INTRODUCTION

Trocar-cannula systems, or trocar-cannulas, can be used to drain fluid from body cavities, introduce fluids into body cavities, and insert tools into body cavities to perform surgical functions. Trocar-cannulas often include two main components: (1) a hollow tube or cannula, and (2) a puncturing member, referred to as a trocar. The cannula is inserted through a wall of a body cavity after the trocar is utilized to puncture the wall.

Trocar-cannulas may be used in cardiovascular surgery, laparoscopic surgery, arthroscopic surgery, and intraocular surgery. In intraocular surgery, for example, a trocar-cannula is often used to obtain access to the posterior-segment of the eye, or the area behind the lens. Typically, a needle or blade of the trocar is removably disposed through the cannula for insertion into the eye. The blade is used to penetrate the eye and insert the cannula. Upon insertion of the cannula, the trocar can be removed from the eye while the cannula remains inserted in the eye.

### SUMMARY

Embodiments of the present disclosure provide devices for inserting one or more trocar-cannulas into bodily tissues.

In certain embodiments, a cannula insertion system is provided, comprising: a housing comprising a proximal end and a distal end, the distal end of the housing comprising an opening; a plurality of shafts disposed within the housing, each of the plurality of shafts comprising a proximal end and a distal end; and a blade disposed at the distal end of each of the plurality of shafts, wherein: each of the plurality of shafts is configured to axially translate between a retracted position and an extended position; the blade at the distal end of each of the plurality of shafts extends beyond the opening of the housing when the respective shaft of the plurality of shafts is in the extended position; and the distal end of each of the plurality of shafts is further configured to support a cannula around the blade.

In certain embodiments, a cannula insertion system is provided, comprising: a housing comprising a proximal end and a distal end, the distal end of the housing comprising an opening; a first shaft disposed within the housing, the first shaft comprising a proximal end and a distal end; a blade disposed at the distal end of the first shaft; a plurality of second shafts disposed within the housing, each of the plurality of shafts comprising a proximal end and a distal end; and a plurality of compartments formed in the housing, each of the plurality of compartments configured to retain a cannula, wherein: the first shaft is configured to axially translate between a retracted position and an extended position; the blade at the distal end of the first shaft extends beyond the opening of the housing when the first shaft is in the extended position; each of the plurality of second shafts is configured to axially translate to an activated position; and in the activated position, each of the plurality of second shafts is configured to release the cannula from a respective compartment of the plurality of compartments for loading the cannula onto the blade at the distal end of the first shaft.

In certain embodiments, a cannula insertion system is provided, comprising: a housing comprising a proximal end and a distal end; a shaft disposed within the housing, the shaft comprising a proximal end and a distal end; a blade disposed at the distal end of the shaft; and a cylinder disposed at the distal end of the housing, the cylinder comprising a plurality of compartments, wherein: each of the plurality of compartments is configured to retain a cannula; the cylinder is configured to rotate about an axis and to a plurality of rotational positions; one of the plurality of compartments is aligned with the shaft in each of the plurality of rotation positions of the cylinder; the shaft is configured to axially translate between a retracted position and an extended position; and in the extended position, the shaft is configured to extend through the one of the plurality of compartments aligned with the shaft for loading and inserting the cannula.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is a side view of an apparatus for inserting cannulas, according to certain embodiments of the present disclosure.

FIG. 1B is a side cross-sectional view of the apparatus of FIG. 1A, according to certain embodiments of the present disclosure.

FIG. 1C is an enlarged side cross-sectional view of a portion of the apparatus of FIG. 1A, according to certain embodiments of the present disclosure.

FIG. 1D is an enlarged side cross-sectional view of a portion of the apparatus of FIG. 1A, according to certain embodiments of the present disclosure.

FIG. 1E is a top-down cross-sectional view of a portion of the apparatus of FIG. 1A, according to certain embodiments of the present disclosure.

FIG. 1F is a top-down cross-sectional view of a portion of the apparatus of FIG. 1A, according to certain embodiments of the present disclosure.

FIG. 2A is a side view of an apparatus for inserting cannulas, according to certain embodiments of the present disclosure.

FIG. 2B is a side cross-sectional view of the apparatus of FIG. 2A, according to certain embodiments of the present disclosure.

FIG. 2C is an enlarged side cross-sectional view of a portion of the apparatus of FIG. 2A, according to certain embodiments of the present disclosure.

FIG. 3A is a side view of an apparatus for inserting cannulas, according to certain embodiments of the present disclosure.

FIG. 3B is a side cross-sectional view of the apparatus of FIG. 3A, according to certain embodiments of the present disclosure.

FIG. 3C is a top cross-sectional view of a portion of the apparatus of FIG. 3A, according to certain embodiments of the present disclosure.

FIG. 4A is a side view of an apparatus for inserting cannulas, according to certain embodiments of the present disclosure.

FIG. 4B is a side cross-sectional view of the apparatus of FIG. 4A, according to certain embodiments of the present disclosure.

FIG. 5 is a side view of an apparatus for inserting cannulas, according to certain embodiments of the present disclosure

FIG. 6A is a side perspective view of an apparatus for inserting cannulas, according to certain embodiments of the present disclosure.

FIG. 6B is a side perspective view of a portion of the apparatus of FIG. 6A, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure provide devices for inserting one or more trocar-cannulas into bodily tissues.

In ophthalmic surgery, and in other surgical operations, surgeons often insert cannulas into surgical openings. In ophthalmic surgeries, such surgical openings are typically formed through the sclera of the eye and are known as sclerotomies. Generally, a cannula can comprise a cannula hub and a cannula tube coupled to and extending from the cannula hub. The cannula hub can be used to hold the cannula against the surface of a tissue, such as the sclera, and facilitate the insertion of a surgical instrument into the cannula tube. Meanwhile, the cannula tube can be a flexible or rigid tube that is inserted into the surgical opening to allow and guide surgical instruments into an interior body portion of a patient.

In many instances, the cannula is inserted into the surgical opening manually by a surgeon using a handheld tool having a handle and a blade. Such a tool is called a trocar. A cannula may be placed on the blade of the trocar, and the surgeon, holding the handle, can insert the blade into the sclera and push the blade into the eye until the cannula is positioned within the resulting sclerotomy. Thereafter, the surgeon may withdraw the blade from the cannula, thereby allowing the surgeon to insert another surgical instrument into the cannula, and/or allowing the cannula to be used as a valve in certain instances.

In ophthalmic surgeries, and in other surgical operations, multiple cannulas are often used simultaneously. For example, in many vitreoretinal procedures, a three-port (e.g., three-cannula) system is used to facilitate simultaneous infusion of fluids, illumination of a target site, and manipulation of target tissues at the target site. Often, the multiple cannulas are pre-assembled on separate, single-use trocars for insertion into the body. However, the utilization of separate trocars can create unnecessary clutter in the surgical environment and compels additional hand-offs between a surgeon and other surgical staff. And, where a single trocar is used with multiple cannulas, the loading of each cannula onto the trocar for insertion can add inefficiencies and additional steps to the surgical procedure.

There is therefore a need for the cannula insertion systems as disclosed herein, which can allow for dispensing and/or insertion of multiple cannulas.

Examples will now be described relative to the Drawings.

As used herein, the term “proximal” refers to a location with respect to a device or a portion of the device that, during normal use, is closest to the user using the device and farthest from the patient in connection with whom the device is used. Conversely, the term “distal” refers to a location with respect to the device or the portion of the device that, during normal use, is farthest from the user using the device and closest to the patient in connection with whom the device is used. For example, the terms “distal” and “proximal” as used herein may refer to a relative location with respect to an insertion device, or a portion thereof.

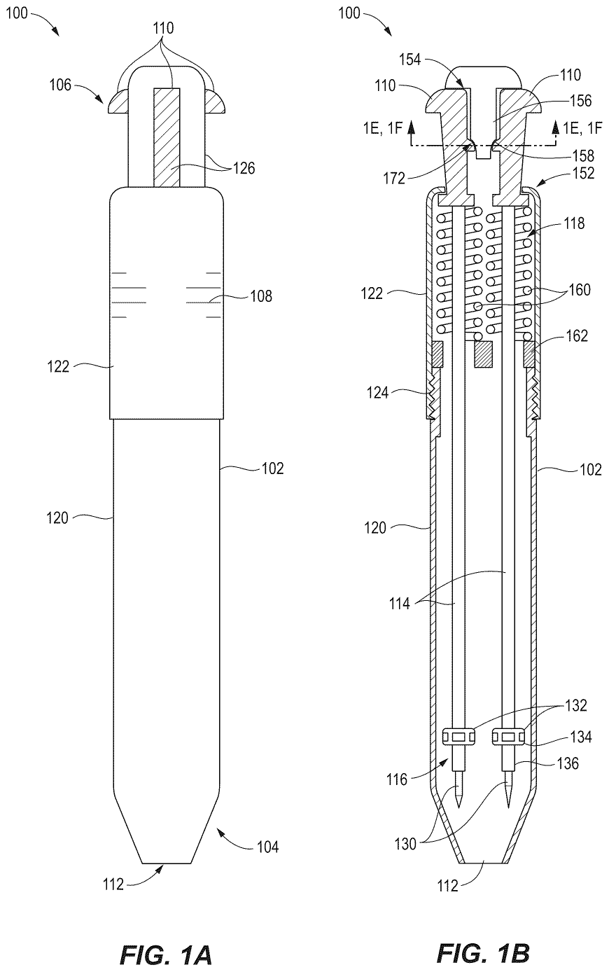

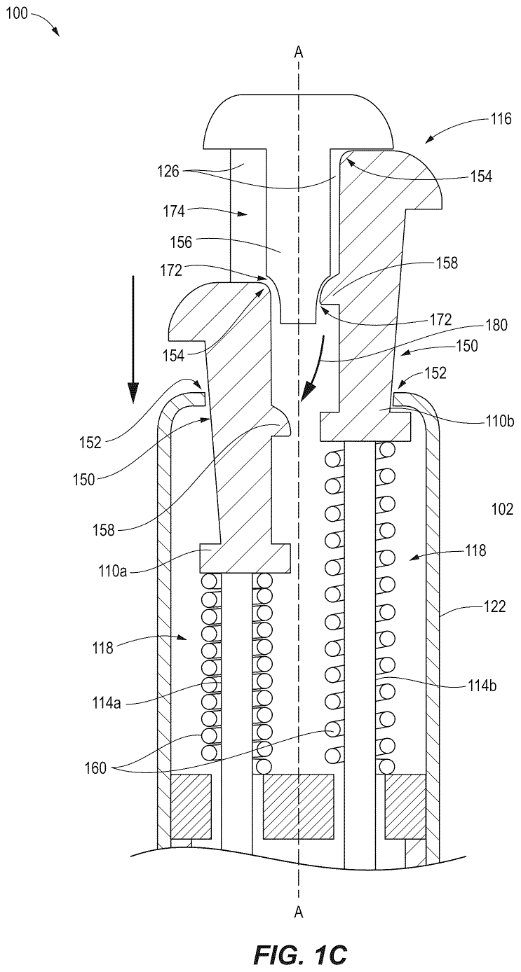

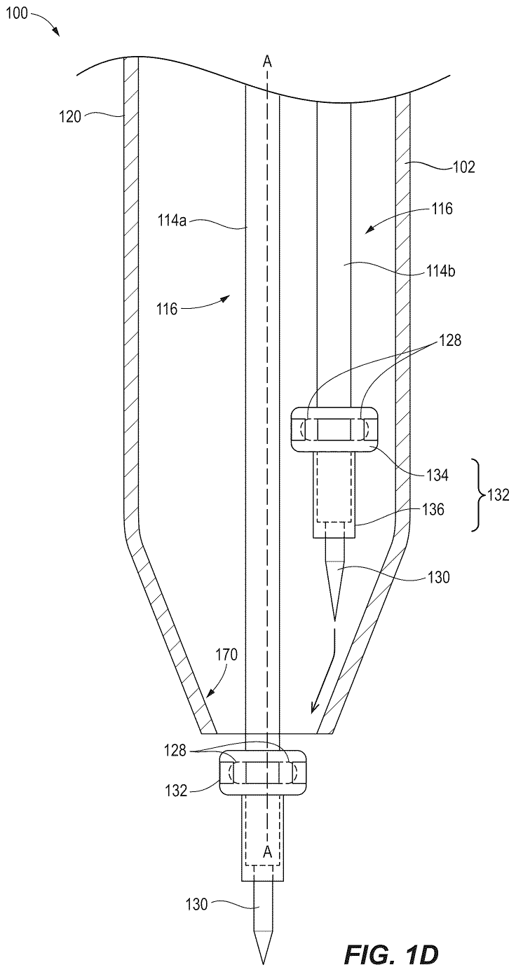

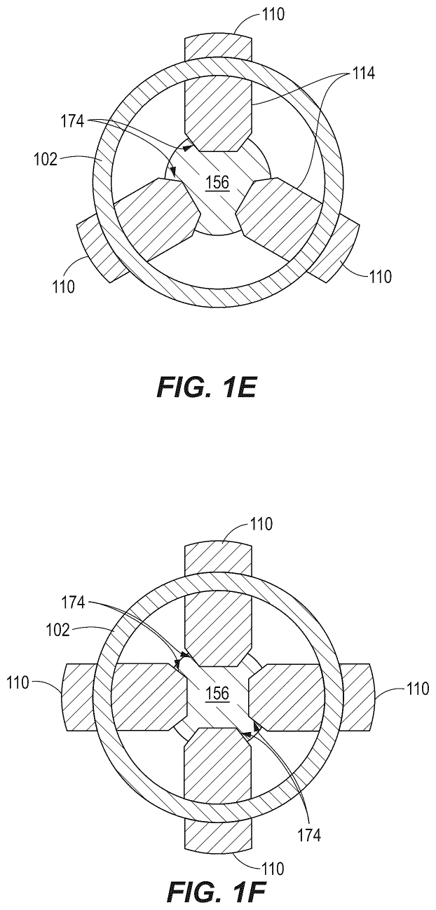

FIG. 1A is a side view of a cannula insertion device 100, according to certain embodiments of the present disclosure. FIG. 1B is a side cross-sectional view of the cannula insertion device 100, according to certain embodiments of the present disclosure. FIGS. 1C and 1D are enlarged side cross-sectional views of the proximal and distal ends of the cannula insertion device 100, respectively, according to certain embodiments of the present disclosure. FIGS. 1E and 1F are top cross-sectional views of a portion of the cannula insertion device 100, according to certain embodiments of the present disclosure. For clarity, FIGS. 1A-1F will be described together herein.

With reference to FIGS. 1A and 1B, the cannula insertion device 100 includes a housing 102 configured to be grasped by a hand of a surgeon or surgical assistant during a surgical operation, such as a vitreoretinal procedure or other ophthalmic procedure. In certain embodiments, the housing 302 may be referred to as a “handle.” In the embodiments of FIGS. 1A-1F, the housing 102 has a generally elongated and cylindrical shape, though other shapes are also contemplated. In certain embodiments, the housing 102 includes one or more textured or contoured surfaces 108 for improved gripping thereof by the user. The housing 102 is formed of any suitable material, and by any suitable method, such as, injection molding or machining. In certain embodiments, the housing 102 is formed of a thermoplastic or metallic material configured to be sterilized and used in a surgical setting. In certain embodiments, the housing 102 is formed of a transparent or translucent material to facilitate visibility of internal components by a user (for example, to see how many cannulas are disposed within the cannula insertion device 100, and the like). In other embodiments, the housing 102 is formed of an opaque material.

In certain embodiments, the housing 102 is formed of multiple segments or components affixed or attached together. For example, in the embodiments of FIGS. 1A and 1B, the housing 102 includes a distal segment 120 and a proximal segment 122 affixed to each other by a threaded connection 124 (e.g., the distal segment 120 and the proximal segment 122 have complementary threads configured to rotatably mate with each other). However, any suitable number of components and types of connections are contemplated.

The housing 102 includes a distal end 104 and a proximal end 106. An opening 112 is disposed at the distal end 104 through which cannulas may be dispensed from the cannula insertion device 100. Meanwhile, a plurality of manual control features are disposed at the proximal end 106 for controlling the administration of cannulas from the cannula insertion device 100. In the embodiments of FIGS. 1A and 1B, the control features include sliding toggles 110, which can also be referred to as “sliding buttons” or simply “sliders,” that are configured to be linearly translated along slots 126 formed at the proximal end 106 of the housing 102. However, such manual control features are only exemplary, and other manual control features may also be used.

Referring now to FIG. 1B, each of the sliding toggles 110 is coupled with a proximal end 118 of one of a plurality of shafts 114 that extends through the housing 102. Generally, each of the plurality of shafts 114 includes an elongated rod-like structure extending between the proximal end 106 and the distal end 104 of the housing 102. A blade 130 is disposed at a distal end 116 of each of the plurality of shafts 114. Each of the blades 130 generally comprises a sharpened, needle-like structure configured to puncture a target tissue of a patient for cannula insertion, such as the sclera of the patient's eye. In certain embodiments, one or more of the blades 130 is monolithically formed with the respective shaft 114. In other words, the blade(s) 130 and the respective shaft(s) 114 are formed of the same material. In other embodiments, however, one or more of the blades 130 and the respective shaft(s) are formed of different materials and are coupled together by any suitable means, such as by an adhesive, overmolding, and the like. In certain embodiments, the blades 130 and/or shafts 114 are formed of a thermoplastic or metallic material configured to be sterilized and used in a surgical setting.

In certain embodiments, the proximal end 118 of each shaft 114 has a biasing device 160 disposed therearound. The biasing device 160 extends between the respective sliding toggle 110 coupled to the shaft 114 and a biasing surface 162 of the housing 102, which may be disposed within the proximal segment 122. During use, the biasing device 160 imparts proximal force(s) against the sliding toggle 110, and therefore, the shaft 114 coupled thereto, to bias the sliding toggle 110 and the shaft 114 proximally. As further described below, proximal biasing by the biasing device 160 facilitates return of each sliding toggle 110 and respective shaft 114 to a “retracted” or “storage” position. Generally, the biasing device 160 may include any suitable type of a biasing device 160, such as a coil spring, flat spring, and the like.

Each of the shafts 114 is further configured to support, or retain, a cannula 132 around the respective blade 130 at the distal end 116 for insertion. For example, each of the shafts 114 may include one or more retaining features 138 (shown in phantom in FIG. 1D) formed thereon, including grooves, protrusions, and the like, configured to engage with one or more complementary features on the respective cannula 132 to temporarily retain the cannula 132. The complementary features on the cannulas 132 may be formed on either a hub 134 or a tube 136 of the cannulas 132.

Referring now to FIGS. 1C and 1D, manual actuation of each sliding toggle 110 along or within the respective slot 126 causes corresponding linear translation of the respective shaft 114 through the housing 102. Generally, each sliding toggle 110, and therefore, each respective shaft 114 and blade 130, can be actuated between the retracted position and an “extended” position. In FIG. 1C, the sliding toggle 110a on the right is shown in the extended position, and the sliding toggle 110b on the left is shown in the retracted position. In the extended position, the sliding toggle 110a is disposed at a proximal position within the slot 126. In the retracted position, the sliding toggle 110b is disposed at a distal position in the slot 126. Similarly in FIG. 1D, the shaft 114a on the left is shown in the extended position, and the shaft 114b on the right is shown in the retracted position.

In the retracted position, the respective shaft 114 and blade 130 attached thereto are disposed completely within the housing 102. In the extended position, at least the blade 130 and the distal end 116 of the respective shaft 114 are extended beyond the opening 112. Thus, to insert the cannula 132 disposed on one of the plurality of shafts 114, the respective sliding toggle 110 is actuated to the extended position such that blade 130 and the cannula 132 of the shaft 114 are extended through the opening 112 of the housing 102, therefore allowing the user (e.g., a surgeon) to penetrate the target tissue (e.g., a patient's sclera) with the blade 130 and push the cannula 132 into the resulting opening. Thereafter, the cannula insertion device 100 can be pulled away from the tissue, leaving the cannula 132 in the tissue for subsequent use.

One or more components or surfaces within the housing 102 interface with and guide the shafts 114 and/or sliding toggles 110 within the housing 102 during translation. For example, as shown in FIG. 1C, the housing 102 can include a central support member 156 disposed at the proximal end 106 to stabilize and guide each of the sliding toggles 110 during actuation by the user. The central support member 156 includes guideways 174 (also shown in FIGS. 1E and 1F), such as rails or slots, upon which the sliding toggles 110 can linearly translate during actuation. Additional interfacing surfaces of the housing 102 may include the biasing surface 162, a ledge 152, and an inner surface 170 at the distal end 104 of the housing 102, which are described in further detail below.

In certain embodiments, each sliding toggle 110 may have one or more features to facilitate temporary locking and unlocking of the sliding toggle 110 and respective shaft 114 in the extended position. For example, as shown in FIG. 1C, each toggle 110 has a sloped or curved outer surface 150 that interacts with the ledge 152 of the housing 102 to cause inward displacement of the sliding toggle 110 toward a central axis A of the housing 102 when the toggle is actuated distally. This inward displacement during actuation of the sliding toggle 110 causes a locking surface 154 of the sliding toggle 110 to nestle against a locking structure 172 of the central support member 156, which may have a sloped or curved surface along which the locking surface 154 can slide and catch to lock the sliding toggle 110 in place.

To release the sliding toggle 110 from the extended position (toggle 110A in FIG. 1C), the user may depress another sliding toggle 110 (toggle 110B in FIG. 1C). For example, each sliding toggle 110 may have a release feature 158 formed on and/or extending from a medial surface of the sliding toggle 110 that is configured to interact with, and “bump” outwardly, the release feature 158 of another sliding toggle 110 disposed in the extended position. Thus, a retracted sliding toggle 110 can be actuated toward the extended position to “bump” and move another extended sliding toggle 110 away from the central support member 156, as represented by the arrow 180 in FIG. 1C, after which the biasing device 160 will cause return of the sliding toggle 110 and respective shaft 114 to the retracted position.

With reference to FIG. 1D, in certain embodiments, the distal end 104 of the cannula insertion device 100 may include a tapered or angled inner surface 170 that distally terminates at the opening 112. In such embodiments, the inner surface 170 may guide each of the blades 130 and surrounding cannulas 132 toward the opening 112 when the respective shaft 114 is actuated to the extended position. Thus, in the illustrated embodiments, the distal ends 116 of the shafts 114 may be displaced inwardly toward the central axis A by the inner surface 170 when in or actuated to the extended position. Gaps, or tolerances, between the various components within the cannula insertion device 100, such as between the shaft 114 and the biasing surface 162, may be designed or optimized to facilitate such inward displacement of the distal ends 116 without bending the shafts 114.

Referring now to FIGS. 1E and 1F, various exemplary arrangements for the shafts 114 and corresponding sliding toggles 110 are shown. In FIGS. 1E and 1F, which are top-down cross-section views from the proximal end 106 of cannula insertion device 100, the shafts 114 are shown in phantom through the sliding toggles 110. As shown in FIG. 1E, in certain embodiments, the cannula insertion device 100 includes three shafts 114 annularly arranged and evenly spaced within the housing 102. In certain other embodiments, such as shown in FIG. 1F, the cannula insertion device 100 includes four shafts 114 annularly arranged and evenly spaced within the housing 102. However, any number and/or arrangement of the shafts 114 is contemplated by this disclosure to facilitate use of the cannula insertion device 100 in a variety of surgical operations.

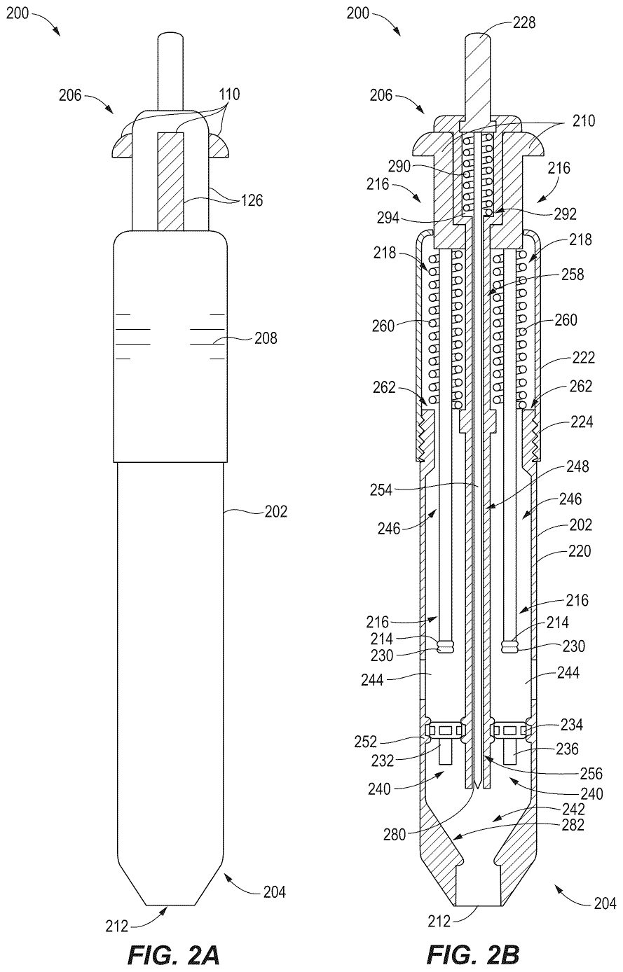

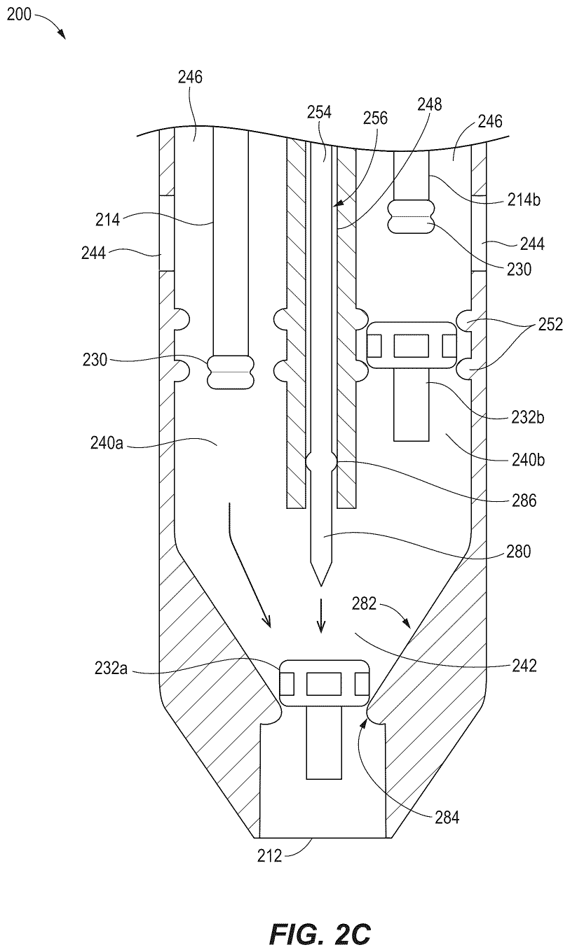

FIG. 2A is a side view of a cannula insertion device 200, according to certain embodiments of the present disclosure. FIGS. 2B and 2C are side cross-sectional views of the cannula insertion device 200, according to certain embodiments of the present disclosure. For clarity, FIGS. 2A-2C will be described together herein.

The cannula insertion device 200 is substantially similar to the cannula insertion device 100. For example, the cannula insertion device 200 includes a housing 202 configured to be grasped by a hand of a surgeon or surgical assistant during a surgical operation, such as a vitreoretinal procedure or other ophthalmic procedure. In certain embodiments, the housing 202 may be referred to as a “handle.” The housing 202 has a generally elongated and cylindrical shape, though other shapes are also contemplated. In certain embodiments, the housing 202 includes one or more textured or contoured surfaces 208 for improved gripping thereof by the user. The housing 202 is formed of any suitable material, and by any suitable method, such as for example, injection molding or machining. In certain embodiments, the housing 202 is formed of a thermoplastic or metallic material configured to be sterilized and used in a surgical setting. In certain embodiments, the housing 202 is formed of a transparent or translucent material to facilitate visibility of internal components by a user (for example, to see how many cannulas are disposed within the cannula insertion device 200, and the like). In other embodiments, the housing 202 is formed of an opaque material.

In certain embodiments, the housing 202 is formed of multiple segments or components affixed or attached together. For example, in the embodiments of FIGS. 2A and 2B, the housing 202 includes a distal segment 220 and a proximal segment 222 affixed to each other by a threaded connection 224 (e.g., the distal segment 220 and the proximal segment 222 have complementary threads configured to rotatably mate with each other). However, any suitable number of components and types of connections are contemplated.

The housing 202 includes a distal end 204 and a proximal end 206. An opening 212 is disposed at the distal end 204 through which cannulas may be dispensed from the cannula insertion device 200. Meanwhile, a plurality of manual control features are disposed at the proximal end 206 for controlling the administration of cannulas from the cannula insertion device 200. In the embodiments of FIGS. 2A and 2B, the control features include sliding toggles 210 that are configured to be linearly translated along slots 226 formed at the proximal end 206 of the housing 202. The control features further include a depressible button 228 at a most proximal position of the proximal end 206. However, such manual control features are only exemplary, and other manual control features may also be used.

Referring now to FIG. 2B, each of the sliding toggles 210 is coupled with a proximal end 218 of one of a plurality of first shafts 214 that extends through the housing 202. Generally, each of the plurality of shafts 214 includes an elongated rod-like structure extending between the proximal end 206 and the distal end 204 of the housing 202. The shafts 114 may be formed of a thermoplastic or metallic material configured to be sterilized and used in a surgical setting. A distal end 216 of each of the plurality of shafts 214 is configured to engage with one of a plurality of cannulas 232 disposed within the housing 202 to transfer the cannula 232 from one of a plurality of storage bays 240 to a loading bay 242 for loading onto a blade. In certain embodiments, a plunger 230 is disposed at the distal end 216 of each shaft 214 for directly contacting and pushing against the hubs 234 of the cannulas 232. In such embodiments, the plungers 230 can formed of an elastomeric material to reduce or prevent damage to the cannulas 232. Examples of suitable elastomeric materials include thermoplastic elastomers configured to be sterilized. The plungers 230 may be coupled to the shafts 214 by any suitable means, as by an adhesive, overmolding, and the like.

In certain embodiments, the cannula insertion device 200 includes three shafts 214 (and thus also, three toggles 210) annularly arranged and evenly spaced within the housing 202. In certain other embodiments, the cannula insertion device 200 includes four shafts 214 (and thus also, four toggles 210) annularly arranged and evenly spaced within the housing 202. However, any number and/or arrangement of the shafts 214 is contemplated by this disclosure to facilitate use of the cannula insertion device 200 in a variety of surgical operations.

In certain embodiments, each shaft 214 is disposed within one of a plurality of distinct chambers 246 arranged within and extending through the housing 202. Such chambers 246 may facilitate guidance of the shafts 214 during actuation thereof. In certain embodiments, however, the plurality of shafts 214 may be arranged around a singular annular chamber 246.

In certain embodiments, the proximal end 218 of each shaft 214 has a biasing device 260 disposed therearound. The biasing device 260 extends between the respective toggle 210 coupled to the shaft 214 and a biasing surface 262 of the housing 202. During use, the biasing device 260 imparts proximal force(s) against the sliding toggle 210, and therefore, the shaft 214 coupled thereto, to bias the sliding toggle 210 and the shaft 214 proximally. Proximal biasing by the biasing device 260 facilitates return of each sliding toggle 210 and respective shaft 214 to a “retracted” or “storage” position after actuation to an “extended” position. Generally, the biasing device 260 may include any suitable type of a biasing device 260, such as a coil spring, flat spring, and the like.

Distal to each shaft 214 within the housing 102 is one of the plurality of storage bays 240. Each storage bay 240 generally includes a structure, or a chamber, for storing one of the plurality of cannulas 232. For example, in the embodiments of FIGS. 2B and 2C, the storage bays 240 protrusions 252, such as knobs or ledges, upon which the hubs 234 of the cannulas 232 can be suspended within the storage bays 240. Generally, a cannula 232 may be “pushed” out of a storage bay 240 upon application of sufficient distal force(s) by the respective shaft 214 when a user actuates the corresponding toggle 210. In embodiments where each shaft 214 is disposed through a distinct and separate chamber 246, the storage bays 240 may each be disposed within, or along, a chamber 246 of a respective shaft 214. In certain embodiments, the cannulas 232 may be loaded into the storage bays 240 prior to assembly of the distal segment 220 and proximal segment 222 by inserting the cannulas 232 through the proximal end of the distal segment 220. In certain embodiments, the cannulas 232 may be loaded into the storage bays 240 via ports 244 disposed through a sidewall of the housing 202.

Similar to the sliding toggles 210, the button 228 is coupled with a proximal end 258 of a second shaft 254 that extends through the housing 202. Generally, the shaft 254 includes an elongated rod-like structure extending between the proximal end 206 and the distal end 204, similar to the shafts 214. And similarly, the shaft 254 may be formed of a thermoplastic or metallic material configured to be sterilized and used in a surgical setting. However, unlike the shafts 214, a distal end 256 of the shaft 254 has a blade 280 attached or formed thereon. The blade 280 generally comprises a sharpened, needle-like structure configured to puncture a target tissue of a patient for cannula insertion, such as the sclera of the patient's eye. In certain embodiments, the blade 280 is monolithically formed with the shaft 254 (for example, formed of the same material). In other embodiments, however, the blade 280 and the shaft 254 are formed of different materials and are coupled together by any suitable means, such as by an adhesive, overmolding, and the like. In certain embodiments, the blade 280 is formed of a thermoplastic or metallic material.

The shaft 254 is further configured to support, or retain, one of the cannulas 232 around the blade 280 at the distal end 256 for insertion. For example, the shaft 254 may include one or more retaining features 286 (shown in FIG. 2C, and similar to those shown in phantom in FIG. 1D) formed thereon, including grooves, protrusions, and the like, configured to engage with one or more complementary features on the cannula 232 to temporarily retain the cannula 232. The complementary features on the cannula 232 may be formed on either the hub 234 or a tube 236 of the cannula 232.

In certain embodiments, the shaft 254 is disposed within an individual chamber 248 arranged within and extending through the housing 202. The chamber 248 may facilitate guidance of the shaft 254 during actuation thereof. In the embodiments of FIGS. 2B and 2C, the chamber 248 is centrally disposed within the housing 202, with the chamber(s) 246 disposed around the chamber 248.

In certain embodiments, the proximal end 258 of the shaft 254 has a biasing device 290 disposed therearound. The biasing device 290 extends between the button 228 and a biasing surface 292 of the housing 202, which may include a ledge or other protrusion formed between the chamber 248 and a slot or opening 294 configured to receive the button 228 when depressed by a user. During use, the biasing device 290 imparts proximal force(s) against the button 228, and therefore, the shaft 254 coupled thereto, to bias the button 228 and the shaft 254 proximally. Proximal biasing by the biasing device 290 facilitates return of each button 228 and the shaft 254 to a “retracted” position after actuation to an “extended” position. Generally, the biasing device 290 may include any suitable type of a biasing device, such as a coil spring, flat spring, and the like.

Distal to the shafts 214 and 254, and their corresponding chambers 246 and 248, respectively, is the loading bay 242. The loading bay 242 includes a space, or chamber, within the cannula insertion device 200 configured to receive a cannula 232 transferred therein from one of the storage bays 240 via actuation of the respective shaft 214. Once the cannula 232 is positioned within the loading bay 242, the cannula 232 may be loaded onto the blade 280 and inserted into the target tissue.

Referring now to FIG. 2C, the distal end 204 of the housing 202 during loading of one of the cannulas 232 onto the blade 280 is illustrated. As shown, the cannula 232a has been released from, or pushed out of, its storage bay 240a and into the loading bay 242 by actuation of the respective shaft 214a to the extended position. Meanwhile, the cannula 232b remains suspended within its storage bay 240b, as the shaft 214b has not been distally actuated by the user.

In FIG. 2C, the loading bay 242 comprises a sloped surface 282 and a lip 284 proximal to the opening 212. The surface 282 functions to direct or guide the released cannula 232a to the lip 284, which the cannula 232a may rest upon while being loaded onto the blade 280. Generally, the lip 284 is positioned such as to be concentric with a central axis B of the housing 202, shaft 254, and/or blade 280, thereby enabling the blade 280 to pass through the cannula 232a when the cannula 232a is positioned on the lip 284 and the shaft 254 is distally translated. Accordingly, after the cannula 232a is positioned on the lip 284, the user may then depress the button 228 to distally actuate the shaft 254 to the extended position, causing the blade 280 to pass through the cannula 232a. As the blade 280 passed through the cannula 232a, the retaining features 286 “catch” complementary features on the cannula 232a, thereby loading it onto the blade 280. When sufficient distal force(s) are exerted upon the button 228 by the user, the cannula 232a is pushed beyond the lip 284 and through the opening 212 (while loaded on the blade 280) to the exterior of the housing 202 for insertion into a tissue. In certain embodiments, loading the cannula 232a onto the blade 280 and extending the blade 280 through the opening 212 may be carried out in a singular, depressing motion against the button 228 by the user.

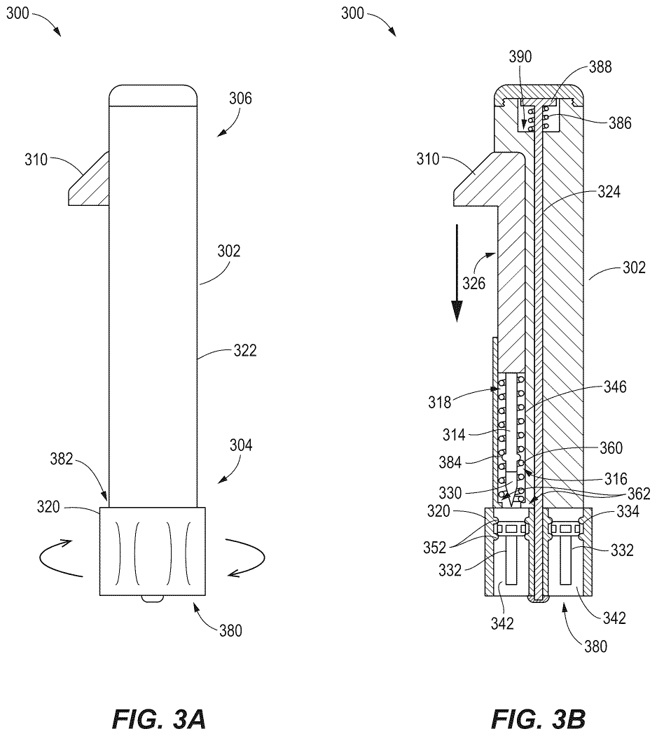

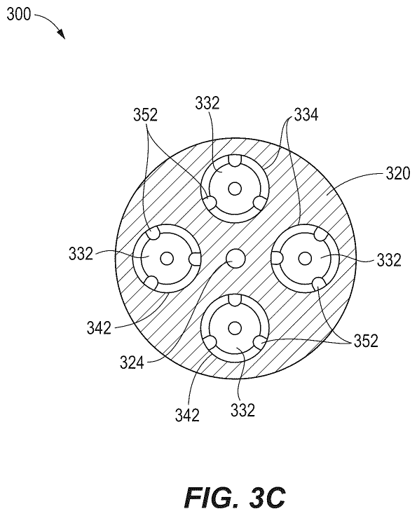

FIG. 3A is a side view of a cannula insertion device 300, according to certain embodiments of the present disclosure. FIGS. 3B and 3C are side cross-sectional and top cross-sectional views of the cannula insertion device 300, respectively, according to certain embodiments of the present disclosure. For clarity, FIGS. 3A-3C will be described together herein.

The cannula insertion device 300 includes a housing 302. The housing 302 has a generally elongated and cylindrical shape, though other shapes are also contemplated. The housing 302 is formed of any suitable materials, and by any suitable method, such as for example, injection molding or machining. In certain embodiments, the housing 302 is formed of a thermoplastic or metallic materials configured to be sterilized and used in a surgical setting. In certain embodiments, the housing 302 is formed of a transparent or translucent material to facilitate visibility of internal components by a user. In other embodiments, the housing 302 is formed of an opaque material.

The housing 302 includes a handle 322 configured to be grasped by a hand of a surgeon or surgical assistant during a surgical operation, such as a vitreoretinal procedure or other ophthalmic procedure. In certain embodiments, the housing 302 may include one or more textured or contoured surfaces for improved gripping thereof by the user. A distal end 304 of the handle 322 is rotatably coupled to a cylinder 320, while a proximal end 306 of the handle includes one or more manual control features for controlling the administration of cannulas from the cannula insertion device 300. In the embodiments of FIGS. 3A and 3B, the control feature(s) include a sliding toggle 310 that is configured to be linearly translated along a slot 326 formed at the proximal end 306 of the handle 322. However, such a manual control feature is only exemplary, and other manual control features may also be used.

Referring now to FIG. 3B, the sliding toggle 310 is coupled with a proximal end 318 of a shaft 314 that extends through the handle 322. Generally, the shaft 314 includes an elongated rod-like structure extending between the proximal end 306 and the distal end 304 of the handle 322. The shaft 314 may be formed of a thermoplastic or metallic material configured to be sterilized and used in a surgical setting. A blade 330 is disposed at a distal end 316 of the shaft 314. The blade 330 generally comprises a sharpened, needle-like structure configured to puncture a target tissue of a patient for cannula insertion, such as the sclera of the patient's eye. In certain embodiments, the blade 330 is monolithically formed of the same material with the shaft 314. In other embodiments, however, the blade 330 and the shaft 314 are formed of different materials and are coupled together by any suitable means, such as by an adhesive, overmolding, and the like. In certain embodiments, the blade 330 is formed of a thermoplastic or metallic material configured to be sterilized and used in a surgical setting.

The shaft 314 is further configured to temporarily support, or retain, a cannula around the blade 330 at the distal end 316 for insertion. For example, the shaft 314 may include one or more retaining features (similar to those shown in phantom in FIG. 1D) formed thereon, including grooves, protrusions, and the like, configured to engage with one or more complementary features on a cannula to temporarily retain the cannula. The complementary features on the cannula may be formed on either the hub or the tube of the cannula.

In certain embodiments, the shaft 314 and blade 330 are movably disposed within a compartment 346 arranged within and extending through the handle 322. The compartment 346 facilitates guidance of the shaft 314 during actuation thereof and may be at least partially defined by one or more surfaces configured to interface with the shaft 314. In certain embodiments, a biasing device 360 is disposed within the compartment 346 and around the proximal end 318 of the shaft 314. The biasing device 360 extends between the sliding toggle 310 coupled to the shaft 314 and a biasing surface 362 within the compartment 346. During use, the biasing device 360 imparts proximal force(s) against the sliding toggle 310, and therefore, the shaft 314 coupled thereto, to bias the sliding toggle 310 and the shaft 314 proximally. As further described below, proximal biasing by the biasing device 360 facilitates return of the sliding toggle 310 and shaft 314 to a “retracted” position. Generally, the biasing device 360 may include any suitable type of a biasing device, such as a coil spring, flat spring, and the like.

The cylinder 320 comprises a plurality of chambers 342 extending from a proximal end 382 of the cylinder 320 to a distal end 380 of the cylinder 320. Each chamber 342 is sized and arranged to receive and retain each cannula 332 for insertion. Accordingly, each chamber 342 includes a structure for retaining the cannula 332 inserted into the chamber 342. For example, in the embodiments of FIGS. 3B and 3C, the chamber 342 include protrusions 352 upon which hubs 334 of the cannulas 332 can be suspended within the chamber 342. Generally, a cannula 332 may be “pushed” out of, and/or inserted into, a chamber 342 upon application of sufficient force(s) to overcome the retaining force(s) of the protrusions 352.

In certain embodiments, the cylinder 320 includes three chambers 342 annularly arranged and evenly spaced within the cylinder 320. In certain other embodiments, as shown in FIG. 3C, the cylinder 320 includes four chambers 342 annularly arranged and evenly spaced within the cylinder 320. However, any number and/or arrangement of the chambers 342 is contemplated by this disclosure to facilitate use of the cannula insertion device 300 in a variety of surgical operations.

The cylinder 320 is formed of any suitable material, and by any suitable method, such as for example, injection molding or machining. In certain embodiments, cylinder 320 is formed of a thermoplastic or metallic material configured to be sterilized and used in a surgical setting. In certain embodiments, the cylinder 320 is formed of a transparent or translucent material to facilitate visibility into the chambers 342 and see how many cannulas 332 are disposed therein. In other embodiments, the cylinder 320 is formed of an opaque material.

The cylinder 320 is rotatably coupled to the distal end 304 of the handle 322 by a pin 324. The cylinder 320 may be configured to rotate about a central axis “C” of the pin 324, which in certain embodiments, may be aligned with a central axis of the handle 322. In certain embodiments, rotation may be manually driven by a user. In certain embodiments, the cannula insertion device 300 may further include an electromechanical motor or other similar device to electromechanically drive rotation of the cylinder 320, which can be controlled via a user input interface (such as a mechanical toggle).

The cylinder 320 can be rotated to align one of the chambers 342 with the compartment 346 to facilitate the loading of the cannula 332 within the chamber 342 onto the blade 330 for insertion into a target tissue. For example, to insert a desired cannula 332 into the target tissue, a user may first rotate the cylinder 320 about the central axis C to align the respective chamber 342 retaining the cannula 332 with the compartment 346. The user may then actuate the sliding toggle 310 toward the extended position such that blade 330 is distally extended through the chamber 342 to load the cannula 332 onto the blade 330. Generally, “loading” of a cannula 332 may include actuating the blade 330 through the cannula 332 such that one or more retaining features on the shaft 314 or blade 330 engage with one or more complementary features on the cannula 332 to temporarily retain the cannula around the blade 330. After loading, the user may actuate the sliding toggle 310 to a most distal position to cause the blade 330 to extend beyond the distal end 380 of the cylinder 320, thereby allowing the user (e.g., a surgeon) to penetrate the target tissue (e.g., a patient's sclera) with the blade 330 and push the cannula 332 into the resulting opening. The cannula insertion device 300 can then be pulled away from the tissue, leaving the cannula 332 in the tissue for subsequent use. In certain embodiments, loading the cannula 332 onto the blade 330 and extending the blade 330 beyond the distal end 380 may be carried out in a singular, distal sliding motion of the sliding toggle 310 by the user.

In certain embodiments, the cylinder 320 can be locked in place when a chamber 342 is aligned with the compartment 346. For example, in certain embodiments, the distal end 304 of the handle 322 includes one or more locking features 384 configured to engage with a complementary feature on the proximal end 382 of the cylinder 320, or vice versa, when a chamber 342 is aligned with the compartment 346. Such locking features 384 may include protrusions, knobs, grooves, slots, and the like. During use, a user may pull or push the cylinder 320 distally to unlock the cylinder 320 and enable rotation thereof for alignment of a desired chamber 342 with the compartment 346. A biasing device 386 is disposed around and acts proximally upon the pin 324 to facilitate automatic return of the cylinder 320 to the “locked” position once a desired chamber 342 is aligned with the compartment 346. In the example of FIG. 3B, the biasing device 386 is disposed around the pin 324 at the proximal end 306 of the handle 322 and rests on a biasing surface 390 to impart proximal biasing force(s) on a flange 388 or other feature fixedly attached to the pin 324. When the cylinder 320 is pulled or pushed distally by the user, the flange 388 is configured to move proximally through the handle 322 and compress the biasing device 386. Release of the cylinder 320 enable the biasing device 386 to decompress and move the flange 388 and pin 324 proximally. The biasing device 386, like the biasing device 360, may include any suitable type of a biasing device, such as a coil spring, flat spring, and the like.

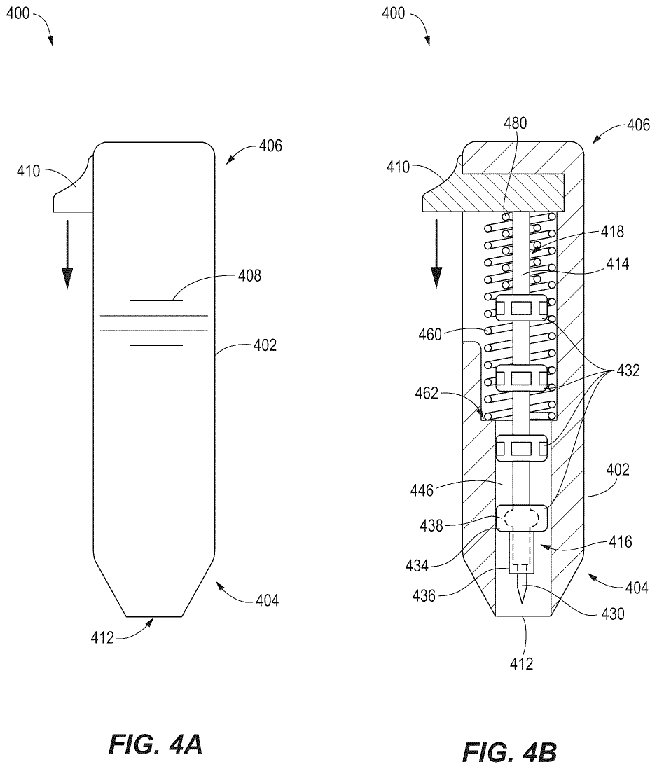

FIG. 4A is a side view of a cannula insertion device 400, according to certain embodiments of the present disclosure. FIG. 4B is a side cross-sectional view of the cannula insertion device 400, according to certain embodiments of the present disclosure. For clarity, FIGS. 4A and 4B will be described together herein.

The cannula insertion device 400 includes a housing 402 configured to be grasped by a hand of a surgeon or surgical assistant during a surgical operation, such as a vitreoretinal procedure or other ophthalmic procedure. In certain embodiments, the housing 402 may be referred to as a “handle.” The housing 402 has a generally elongated and cylindrical shape, though other shapes are also contemplated. In certain embodiments, the housing 402 includes one or more textured or contoured surfaces 408 for improved gripping thereof by the user. The housing 402 is formed of any suitable material, and by any suitable method, such as for example, injection molding or machining. In certain embodiments, the housing 402 is formed of a thermoplastic or metallic material configured to be sterilized and used in a surgical setting. In certain embodiments, the housing 402 is formed of a transparent or translucent material to facilitate visibility of internal components by a user (for example, to see how many cannulas are disposed within the cannula insertion device 400, and the like). In other embodiments, the housing 402 is formed of an opaque material.

The housing 402 includes a distal end 404 and a proximal end 406. An opening 412 is disposed at the distal end 404 through which cannulas may be dispensed from the cannula insertion device 400. Meanwhile, a manual control feature is disposed at the proximal end 406 for controlling the administration of cannulas from the cannula insertion device 400. In the embodiments of FIGS. 4A and 4B, the control feature includes a sliding toggle 410, or “slider,” that is configured to be linearly translated along a slot 426 formed in the housing 402. However, such manual control feature is only exemplary, and other manual control features may also be used.

Referring now to FIG. 4B, the sliding toggle 410 is coupled with a proximal end 418 of a shaft 414 that extends through the housing 402. The shaft 414 includes an elongated rod-like structure extending between the proximal end 406 and the distal end 404 of the housing 402. The shaft 414 may be formed of a thermoplastic or metallic material configured to be sterilized and used in a surgical setting. A blade 430 is disposed at a distal end 416 of the shaft 414. The blade 430 generally comprises a sharpened, needle-like structure configured to puncture a target tissue of a patient for cannula insertion, such as the sclera of the patient's eye. In certain embodiments, the blade 430 is monolithically formed of the same material with the shaft 414. In other embodiments, however, the blade 430 and the shaft 414 are formed of different materials and are coupled together by any suitable means, such as by an adhesive, overmolding, and the like. In certain embodiments, the blade 430 is formed of a thermoplastic or metallic material configured to be sterilized and used in a surgical setting.

In certain embodiments, the shaft 414 and blade 430 are movably disposed within a compartment 446 arranged within and extending through the housing 402. The compartment 446 facilitates guidance of the shaft 414 during actuation thereof and may be at least partially defined by one or more surfaces configured to interface with the shaft 414. In certain embodiments, a biasing device 460 is disposed within the compartment 446 and around the proximal end 418 of the shaft 414. The biasing device 460 extends between the sliding toggle 410 coupled to the shaft 414 and a biasing surface 462, such as a ledge, within the compartment 446. During use, the biasing device 460 imparts proximal force(s) against the sliding toggle 410, and therefore, the shaft 414 coupled thereto, to bias the sliding toggle 410 and the shaft 414 proximally. Proximal biasing by the biasing device 460 facilitates return of the sliding toggle 410 and shaft 414 to a “retracted” position. Generally, the biasing device 460 may include any suitable type of a biasing device, such as a coil spring, flat spring, and the like.

The single shaft 414 is further configured to support, or retain, a plurality of cannulas 432 along a length of the shaft 414 and around the blade 430. This linear arrangement of cannulas 432 along the shaft 414 enables the sequential deployment and insertion of each of the cannulas 432 during use of the cannula insertion device 400. After a most distal cannula 432 along the shaft 414 has been inserted into a target tissue, the remaining plurality of cannulas 432 are automatically translated distally along the shaft 414, and the cannula 432 immediately proximal to the previously inserted cannula 432 is loaded onto the blade 430 for deployment. As used herein, the “most distal” cannula 432 refers to the cannula 432 located at a most distal position along the shaft 414, such as the cannula 432 loaded onto the blade 430. Conversely, a “most proximal” cannula 432 refers to the cannula 432 located at a most proximal position along the shaft 414.

In certain embodiments, the shaft 414 is sized and/or configured to support three cannulas 432 along the length thereof. In certain other embodiments, as shown in FIG. 4B, the shaft 414 is sized and/or configured to support three cannulas 432 along the length thereof. However, any number of the cannulas 432 is contemplated by this disclosure to facilitate use of the cannula insertion device 400 in a variety of surgical operations.

The distal movement of the cannulas 432 along the shaft 414 is facilitated by a biasing device 480. In the embodiments of FIG. 4B, the biasing device 480 is disposed around the shaft 414 between the most proximal cannula 432 and the sliding toggle 410. However, other arrangements of the biasing device 480 are also contemplated. During use, the biasing device 480 imparts distal force(s) against a hub 434 of the most proximal cannula 432, to bias plurality of linearly arranged cannulas 432 toward the distal end 416 of the shaft 414, and to load the most distal cannula 432 onto the blade 430. Generally, the biasing device 480 may include any suitable type of a biasing device, such as a coil spring, flat spring, and the like

To prevent the plurality of cannulas 432 from sliding off the distal end 416 of the shaft 414 as a result of the distal force(s) provided by the biasing device 480, the shaft 414 may include one or more retaining features 438 (similar to those shown in phantom in FIG. 1D) formed at the distal end 416 and/or the blade 430, including grooves, protrusions, and the like. Such retaining features may be configured to engage with one or more complementary features on the most distal cannula 432 to temporarily retain the most distal cannula 432 around the blade 430 and prevent further movement of the plurality of cannulas 432. The complementary features on the cannula 432 may be formed on either the hub 434 or a tube 436 of the cannula 432. Generally, the retaining features on the shaft 414 may be designed to provide enough resistance to prevent the most distal cannula 432 from being pushed off the shaft 414 by the distal force(s) imparted by the biasing device 480, but to also allow the most distal cannula 432 to be pulled off the shaft 414 by friction force(s) imparted by a target tissue when the cannula 432 is inserted therein.

During use, a user (e.g., a surgeon) actuates the sliding toggle 410 to a most distal position to cause the shaft 414 and blade 430 to translate from the retracted position to the extend position, where the blade 430 extends beyond the opening 412. The user then penetrates the target tissue (e.g., a patient's sclera) with the blade 430 and pushes the most distal cannula 432 into the resulting opening. The cannula insertion device 400 can then be pulled away from the tissue, and frictional force(s) by the target tissue cause the cannula 432 to slide off the blade 430 and remain in the tissue for subsequent use. As the most distal cannula 432 is pulled off the shaft 414, the remaining plurality of cannulas 432 are automatically translated distally along the shaft 414 by the distal force(s) provided by biasing device 480, and the cannula 432 immediately proximal to the previously inserted cannula 432 is loaded onto the blade 430 for subsequent insertion into a target tissue.

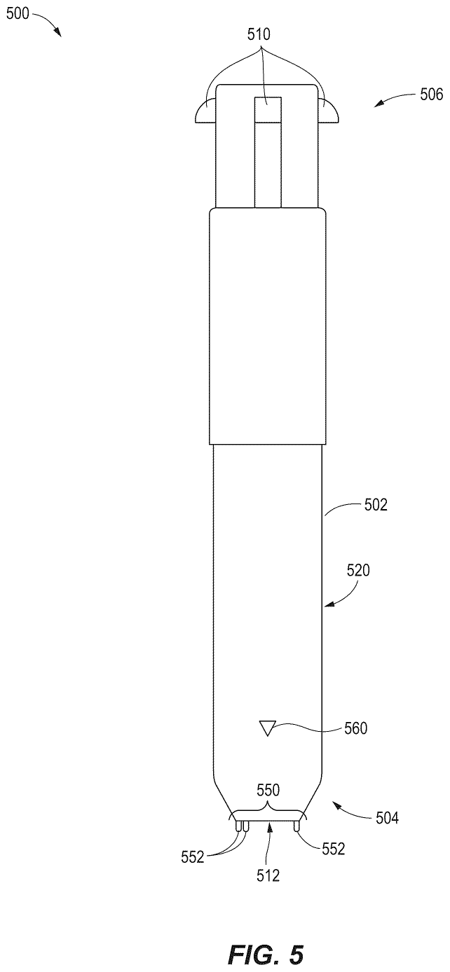

FIG. 5 is a side view of a cannula insertion device 500, according to certain embodiments of the present disclosure. The cannula insertion device 500 is exemplary and can be representative of any of the cannula insertion devices of FIGS. 1-4B.

As shown in FIG. 5, the cannula insertion device 500 includes a housing 502 configured to be grasped by a hand of a surgeon or surgical assistant during a surgical operation, such as a vitreoretinal procedure or other ophthalmic procedure. The housing 502 includes a distal end 504 and a proximal end 506. An opening 512 is disposed at the distal end 504 through which cannulas may be dispensed from the cannula insertion device 100. Meanwhile, a plurality of manual control features 510 are disposed at the proximal end 506 for controlling the administration of cannulas from the cannula insertion device 500.

Also disposed at the distal end 504 of the housing 502 is a marking element 550. The marking element 550 includes one or more marking tips 552 for forming one or more indentations on a target tissue, such as a patient's eye, during a surgical procedure. During use, a user may press the marking element 550 against the target tissue to form indentations thereon and “mark” desired locations of incisions sites for cannula insertion. In the embodiments of FIG. 5, marking element 550 includes three marking tips 552 extending distally from the distal end 504 of the housing 502 and disposed radially outward of the opening 512. However, other numbers and arrangements of marking tips 552 are also contemplated. For example, in certain embodiments, the marking element 550 may include two marking tips 552, four marking tips 552, five marking tips 552, six marking tips 552, or more. Further, the marking element 550 may be arranged at the proximal end 506 of the housing 502 such that the marking tips 552 extend proximally from the proximal end 506, or along a sidewall 520 of the housing 502 between the distal end 504 and the proximal end 506 such that the marking tips 552 extend laterally from the housing 502 (for example, away from a central axis of the housing 502).

The marking element 550 and/or the housing 502 are formed of any suitable material, and by any suitable method, such as for example, injection molding or machining. In certain embodiments, marking element 550 and/or the housing 502 are formed of a thermoplastic or metallic material configured to be sterilized and used in a surgical setting. In certain embodiments, the marking element 550 and/or the housing 502 are formed of the same material; in other embodiments, the marking element 550 and/or the housing 502 are formed of different materials.

Still referring to FIG. 5, in certain embodiments, the cannula insertion device 500 further includes a blade orientation marker 560. The blade orientation marker 560 indicates to the user an orientation of one or more blade(s) disposed within the cannular insertion device 500 to facilitate efficiency during surgical procedures. In certain embodiments, the blade orientation marker 560 is formed on the sidewall 520 of the housing 502. In certain embodiments, the blade orientation marker 560 is formed at the distal end 504 or proximal end 506 of the housing 502. Generally, the blade orientation marker 560 may include any suitable feature for visually indicating the orientation of the blade(s) of the cannula insertion device 500, such as an image or decal on the housing 502, and/or a protrusion or cavity formed in the housing 502.

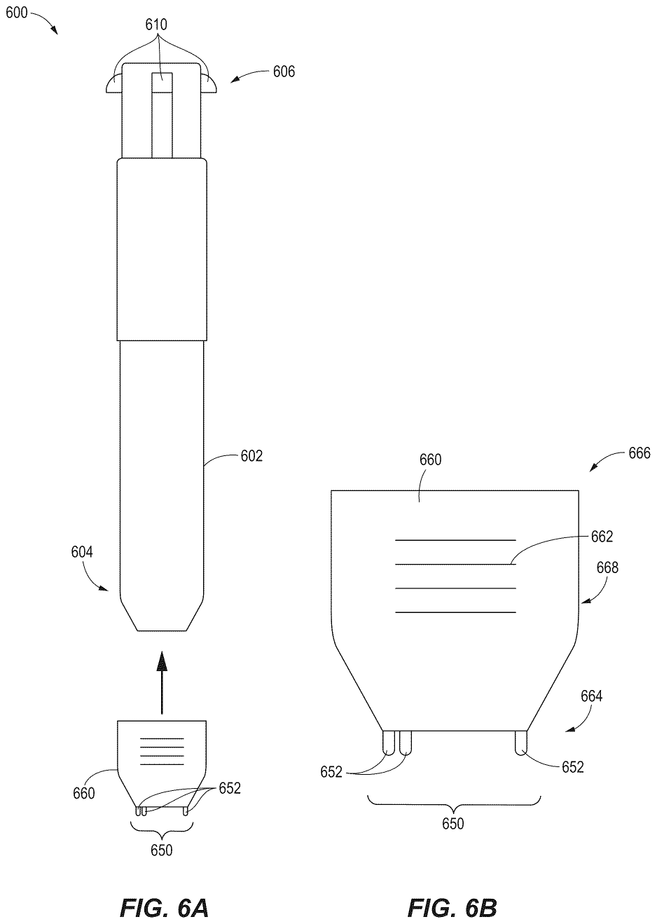

FIG. 6A is a side perspective view of a cannula insertion device 600, according to certain embodiments of the present disclosure. FIG. 6B is a side perspective view of a portion of the cannula insertion device 600, according to certain embodiments of the present disclosure. For clarity, FIGS. 6A and 6B will be described together herein. Note that the cannula insertion device 600 is exemplary and can be representative of any of the cannula insertion devices of FIGS. 1-5.

As shown in FIG. 6A, the cannula insertion device 600 includes a housing 602 configured to be grasped by a hand of a surgeon or surgical assistant during a surgical operation, such as a vitreoretinal procedure or other ophthalmic procedure. The housing 602 includes a distal end 504 and a proximal end 606. An opening 612 is disposed at the distal end 604 through which cannulas may be dispensed from the cannula insertion device 600. Meanwhile, a plurality of manual control features 610 are disposed at the proximal end 606 for controlling the administration of cannulas from the cannula insertion device 600.

Also shown in FIG. 6A is a cap 660. The cap 660 is configured to removably couple to the distal end 504 of the housing 602 to facilitate protection of the components of cannula insertion device 600 during storage, and to facilitate protection of a user handling the cannula insertion device 600 outside of use. For example, the cap 660 may prevent a user from inadvertently actuating the cannula insertion device 600 during handling and injuring themselves with a blade of the cannula insertion device 600.

Turning to FIG. 6B, a magnified view of the cap 660 is shown. In certain embodiments, the cap 660 includes one or more textured or contoured surfaces 662 for improved gripping thereof by the user. In certain embodiments, the cap 660 includes a marking element 650 disposed thereon. The marking element 650 may be substantially similar to the marking element 550 in FIG. 5. Accordingly, during use, a user may press the marking element 650 against the target tissue to form indentations thereon and “mark” desired locations of incisions sites for cannula insertion. In the embodiments of FIG. 6B, the marking element 650 include three marking tips 652 extending distally from a distal end 664 of the cap 660. However, other numbers and arrangements of marking tips 652 are also contemplated. For example, the marking element 650 may include more or less than three marking tips 652, and/or the marking element 650 may be arranged at a proximal end 666 of the cap 660 or along a sidewall 668 of the cap 660 between the distal end 664 and the proximal end 666.

In summary, embodiments of the present disclosure provide cannula insertion systems that allow for efficient dispensing and/or insertion of multiple cannulas.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various embodiments have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus, the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ and the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosure to the specific examples described herein, but rather to also cover all modifications and alternatives coming with the true scope and spirit of the disclosure.

### EXAMPLE EMBODIMENTS

Embodiment 1: A cannula insertion system, comprising a housing comprising a proximal end and a distal end, the distal end of the housing comprising an opening; a shaft disposed within the housing, the shaft comprising a proximal end and a distal end; a blade disposed at the distal end of the shaft; a first biasing device; and a second biasing device, wherein: the shaft is configured to axially translate between a retracted position wherein the blade is disposed within the housing and an extended position wherein the blade extends distally beyond the opening of the housing; the shaft is configured to retain a plurality of cannulas along the shaft in a linear arrangement; the first biasing device is configured to bias the plurality of cannulas towards the blade for loading and insertion of the cannulas; and the second biasing device is configured to bias the shaft toward the proximal end of the housing to return the shaft to the retracted position after insertion of one of the plurality of cannulas.

Embodiment 2: The cannula insertion system of Embodiment 1, wherein the shaft is coupled to a sliding toggle or depressible button at the proximal end of the housing, the sliding toggle or depressible button configured to be manually actuated by a user to actuate the shaft between the retracted position and the extended position.

Embodiment 3: The cannula insertion system of Embodiment 1, wherein, after deployment of a most distal cannula of the plurality of cannulas, a cannula proximal to the deployed most distal cannula is loaded onto the blade for subsequent deployment.

What is claimed is:

1. A cannula insertion system, comprising:
a housing comprising a proximal end and a distal end, the distal end of the housing comprising an opening;
a plurality of shafts disposed within the housing, each of the plurality of shafts comprising a proximal end and a distal end; and
a blade disposed at the distal end of each of the plurality of shafts, wherein:
each of the plurality of shafts is configured to axially translate between a retracted position and an extended position;
the blade at the distal end of each of the plurality of shafts extends beyond the opening of the housing when the respective shaft of the plurality of shafts is in the extended position; and
the distal end of each of the plurality of shafts is further configured to support a cannula around the blade.

2. The cannula insertion system of claim 1, wherein each of the plurality of shafts is coupled to a sliding toggle at the proximal end of the housing, the sliding toggle configured to be manually actuated by a user to actuate the respective shaft of the plurality of shafts between the retracted position and the extended position.

3. The cannula insertion system of claim 2, wherein each sliding toggle is configured to engage with the housing to lock the respective shaft of the plurality of shafts in the extended position upon actuation of the sliding toggle toward the distal end of the housing by the user.

4. The cannula insertion system of claim 3, wherein each sliding toggle is further configured to disengage with the housing to unlock the respective shaft of the plurality of shafts from the extended position upon actuation of the sliding toggle coupled to another one of the plurality of shafts toward the distal end of the housing by the user.

5. The cannula insertion system of claim 2, wherein each of the plurality of shafts or each sliding toggle is coupled to a biasing device, the biasing device configured to bias the respective shaft of the plurality of shafts or the sliding toggle toward the proximal end of the housing to return the respective shaft of the plurality of shafts to the retracted position from the extended position.

6. A cannula insertion system, comprising:
a housing comprising a proximal end and a distal end, the distal end of the housing comprising an opening;
a first shaft disposed within the housing, the first shaft comprising a proximal end and a distal end;
a blade disposed at the distal end of the first shaft;
a plurality of second shafts disposed within the housing, each of the plurality of shafts comprising a proximal end and a distal end; and
a plurality of compartments formed in the housing, each of the plurality of compartments configured to retain a cannula, wherein:
the first shaft is configured to axially translate between a retracted position and an extended position;
the blade at the distal end of the first shaft extends beyond the opening of the housing when the first shaft is in the extended position;
each of the plurality of second shafts is configured to axially translate to an activated position; and
in the activated position, each of the plurality of second shafts is configured to release the cannula from a respective compartment of the plurality of compartments for loading the cannula onto the blade at the distal end of the first shaft.

7. The cannula insertion system of claim 6, wherein releasing the cannula from the respective compartment of the plurality of compartments comprises advancing the cannula into a loading compartment.

8. The cannula insertion system of claim 7, wherein axially translating the first shaft to the extended position after releasing the cannula from the respective compartment of the plurality of compartments causes loading of the cannula onto the blade at the distal end of the first shaft.

9. The cannula insertion system of claim 6, wherein each of the plurality of shafts is coupled to a sliding toggle at the proximal end of the housing, the sliding toggle configured to be manually actuated by a user to actuate the respective shaft of the plurality of shafts between the retracted position and the extended position.

10. The cannula insertion system of claim 9, wherein the first shaft is coupled to a depressible button at the proximal end of the housing, the depressible button configured to be manually actuated by a user to actuate the first shaft between the retracted position and the extended position.

11. A cannula insertion system, comprising:
a housing comprising a proximal end and a distal end;
a shaft disposed within the housing, the shaft comprising a proximal end and a distal end;
a blade disposed at the distal end of the shaft; and
a cylinder disposed at the distal end of the housing, the cylinder comprising a plurality of compartments, wherein:
each of the plurality of compartments is configured to retain a cannula;
the cylinder is configured to rotate about an axis and to a plurality of rotational positions;
one of the plurality of compartments is aligned with the shaft in each of the plurality of rotation positions of the cylinder;
the shaft is configured to axially translate between a retracted position and an extended position; and
in the extended position, the shaft is configured to extend through the one of the plurality of compartments aligned with the shaft for loading and inserting the cannula.

12. The cannula insertion system of claim 11, wherein the cylinder is configured to be manually rotated by a user between the plurality of rotation positions.

13. The cannula insertion system of claim 11, wherein the cylinder is configured to be electromechanically rotated between the plurality of rotation positions.

14. The cannula insertion system of claim 11, wherein the cylinder is configured to be locked in each the plurality of rotation positions.

15. The cannula insertion system of claim 11, wherein the shaft is coupled to a sliding toggle or depressible button at the proximal end of the housing, the sliding toggle or depressible button configured to be manually actuated by a user to actuate the shaft between the retracted position and the extended position.
